(19) United States
(12) Patent Application Publication
(10) Pub. No.: US 2025/0336530 A1
(43) Pub. Date: Oct. 30, 2025

(54) INTER-TRANSFORMATION AND MANAGEMENT SYSTEM OF DIGITAL IMAGING INFORMATION AND CLINICAL DIAGNOSIS INFORMATION AND METHOD THEREOF

(71) Applicants: Jean Fuh, Taipei City (TW); Pei Chin Chen, Taipei City (TW); Martin Mao Tsu Fuh, Taipei City (TW)

(72) Inventors: Jean Fuh, Taipei City (TW); Pei Chin Chen, Taipei City (TW); Martin Mao Tsu Fuh, Taipei City (TW)

(21) Appl. No.: 19/192,149

(22) Filed: Apr. 28, 2025

(30) Foreign Application Priority Data
Apr. 29, 2024 (TW) 113115918

(51) Int. Cl. G16H 50/20; A61B 3/12; G06T 7/00; G06T 7/73

(52) U.S. Cl. CPC G16H 50/20; A61B 3/1233; G06T 7/0016; G06T 7/74; G06T 2207/20081; G06T 2207/30041

(57) ABSTRACT

An inter-transformation and management system and its method for digital imaging information and clinical diagnosis information comprises the following steps: capturing multiple same body part images of an individual in a sequential well defined, regular and predetermined time period by a detecting device to obtain a clinical diagnosis information; performing an individual image processing by an imaging device; performing the identification, confirmation, and inter-transformation procedure by a processing device between the clinical diagnosis information and its pixel digital imaging information; wherein each pixel corresponds to a respective pixel gray level. The processing device compares the changing differences of the pixel gray levels of the previous, sequential and multiple digital imaging information at the same position of the clinical diagnosis information when the changing differences is reaching to a threshold point, the processing device generates a marked signal.

### CROSS-REFERENCE TO RELATED APPLICATION(S)

This application claims the priority benefit of TW application serial No. 113115918 filed on Apr. 29, 2024, the entirety of which is hereby incorporated by reference herein and made a part of the specification.

### BACKGROUND OF THE INVENTION

### Field of the Invention

The invention demonstrates a system and a method for identifying, confirming, inter-transforming, and managing information of a same human body tissue structure which displays distinct information. Particularly, the invention indicates an inter-transformation and management system of a pixel digital imaging information and a clinical diagnosis information, and a method thereof. The inter-transformation and management system and the method thereof are used to process a forward-looking procedure for an individualized medical treatment of an artificial intelligence (AI).

### Description of the Related Art

In recent years, digital information technology of computer science booms and expands to human life. In the clinical medicine, non-infectious chronic diseases illustrate a unique and individualized long-term course in a clinical diagnosis setting.

Taking diabetes as an example: individual patient's fundus retinal vasculature disease causes a vascular rupture/bleeding, resulting in severe visual impairment. The fundus retinal vasculature disease is usually a long-term disease course and lasts for all diabetes patient's life. According to recent studies of the diabetes clinical integrated care program, non-dilated pupil fundus retinal vasculature photography examination once a year can detect an asymptomatic retinal microvascular disease before blood vessel rupture/bleeding (retinal detachment and hemorrhage) causes a severe visual impairment of the patient.

Clinically, in the patient-centered integrated diabetes care program, a regular non-dilated pupil fundus retinal examination once a year allows doctors to detect abnormalities of eye fundus retinal microvascular disease (diabetic fundus retinal microvascular disease) with the professional's naked eye at an early stage. However, regarding the vascular cell tissue of the diabetic fundus retinal microvascular disease, before the doctor first sees the occurrence of the retinal microvascular disease with the naked eye through the non-dilated pupil fundus retinal vasculature photography examination, the cell tissue abnormalities of the diabetic fundus retinal microvascular disease have already occurred.

Therefore, in order to pave the way for the development of artificial intelligence, modern clinical diagnostic instruments or equipment can be used to obtain clinical diagnosis information and corresponding digital imaging information on the changes in the eye fundus retinal microvascular at different time periods with regular intervals using the same non-dilated pupil fundus retinal vasculature photography examination equipment for the same diabetic patient throughout the patient's diabetic life. In clinical practice, the medical examination equipment also comprises radiographic imaging, CT imaging, ultrasound imaging, and the non-mydriatic retinal photography examination of the diabetic patient exemplified in the invention. For clinical physicians (comprising pathologists who are reviewing pathological slides), they obtain the clinical diagnosis information by interpreting the images seen by the naked eye based on their own expertise. For the tissue cells in the same position of the human fundus retinal, the presentation of the digital imaging information and the clinical diagnosis information are completely different. The former (pixel digital imaging information) is obtained by computers through a visible light irradiation, however the computers through the non-visible energy waves and visible light comprise medical equipment also including X-ray radiation and ultrasound. The latter (clinical diagnosis information) is obtained by other way of presentation of the digital image information seen by medical professionals with the naked eye, wherein the digital imaging information is chromatic or achromatic. Both the digital imaging information and the clinical diagnosis information belong to the same human body organ, tissue and cell structures, and obviously the digital imaging information and the clinical diagnosis information are presented in different ways. Therefore, how to identify, confirm, inter-transform and manage the digital imaging information and the clinical diagnosis information will become a topic that needs to be deeply understood between clinical medicine and digital information technology.

### SUMMARY OF THE INVENTION

In view of above mentioned, the invention discloses an inter-transformation and management method of digital imaging information and clinical diagnosis information in the clinical settings, comprising the following steps: capturing multiple same body part images of a human body at each well-defined, regular and predetermined sequential time period, and then obtaining a clinical diagnosis information according to the same body part images by a detecting device; processing an individual image process to obtain the clinical diagnosis information by an imaging device; storing individual digital imaging information by a storage device; automatically identifying, confirming, and inter-transforming the clinical diagnosis information to the digital imaging information by a software program of a processing device; wherein the digital imaging information comprises multiple pixels, and each of the pixels corresponds to a pixel gray level; and processing device would be automatically determining whether any one of changing differences between the pixel gray levels of the pixels at a same position of the digital imaging information of the clinical diagnosis information in the sequential predetermined individual digital imaging information is reaching to a threshold point which would be irreversible by the software program of the processing device; wherein when any one of the changing difference is reaching to the threshold point, the processing device generates a marked signal.

The invention further discloses an inter-transformation and management system for the digital imaging information and its clinical diagnosis information about the diagnosis of clinical medical disease. The system comprises a detecting device, an imaging device, a storage device, and a processing device with a software program. The detecting device captures multiple same body part images of a human body at each predetermined sequential time period, and obtaining clinical diagnosis information of a medical disease according to the same body part images. The imaging device functionally takes an individual's body image process accordingly to obtain the clinical diagnosis information. The storage device is connected to the detecting device and the imaging device and stores individual's sequentially multiple digital imaging information of the same body part images, and multiple pixel gray levels at each predetermined sequential time period. The processing device processes a digital imaging information according to the clinical diagnosis information via a clinical professional's naked eye by an imaging device and then identifies, confirms, and inter-transforms the same position (area) of the clinical diagnosis information to obtain the digital imaging information by a software program. The position (area) of the clinical diagnosis information and its corresponding digital imaging information are obtained in multiple sequential time period of the same body part images of an individual. The digital imaging information comprises multiple pixels and each pixel corresponds to a pixel gray level. The processing device would determine whether any one of changing differences between the pixel gray level at a same position of the sequential digital imaging information of its corresponding clinical diagnosis information is reaching to a threshold point by the software program. Any one of the changing differences in the sequential time period digital imaging information is reaching to a threshold point, the processing device generates a marked signal.

As mentioned above, for the development and the application of the artificial intelligence in the clinical disease diagnosis and treatment, the detection devices such as X-ray (radiology) equipment, ultrasound devices, C-T scanner, MRI machine or computer color photography, the imaging devices, the storage devices stored the digital image information and the processing devices with a software program, and the radiotherapy equipment are connected through the Internet, so that the earliest clinical diagnosis (based on the difference in pixel gray level changes in the digital image information) and treatment methods (radiotherapy/high-energy electron/magnetic wave ablation therapy) are combined into one. Based on the wireless network technology, the inter-transformation and management of the pixel digital image information would be widely used. Through the artificial intelligence and the machine learning, accurate clinical diagnosis can be performed early. According to the earliest clinical diagnosis, the individual is immediately and automatically given the correct, accurate and effective treatment, thereby the early diagnosis and early treatment of human diseases with individualized precision medicine can be achieved.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is the structural block schematic diagram for the inter-transformation and management system of the digital imaging information and the clinical diagnosis information in the invention;

FIG. 2 is the flowchart of the inter-transformation and management of the digital imaging information and the clinical diagnosis information in the invention;

FIG. 3 to FIG. 5 are the sequential clinical diagnosis information diagrams at distinct sequential, well-defined and regular time periods (month/day/yr) of the right eye of the diabetes patient's non-dilated pupil fundus retinography in the invention; A is the point of interest or the area of interest A;

FIG. 6 to FIG. 8 are the diabetes patient's schematic diagrams of the pixel gray level of the digital imaging information of the point of interest A or the area of interest A corresponding to the position of the clinical diagnosis information of fundus retinography at different predetermined time period (month/day/yr); and

FIG. 9 to FIG. 11 are the diabetes patient's schematic diagrams showing the pixel gray levels of the area around the point of interest A corresponding to the position of the clinical diagnosis information of the retinal microvascular lesions illustrated by the processing device using a software program at different time period (month/day/yr).

### DETAILED DESCRIPTION OF THE INVENTION

The invention takes a diabetic fundus retinal microvascular disease as an example. Other detecting devices comprise a mammography detection device, a chest and abdominal radiography detection device, a neck thyroid ultrasound detection device, an abdomen and pelvic ultrasound detection device, a brain CT scan detection device, and other various detection devices that can obtain images. The clinical diagnosis information is established by clinical medical professionals based on their naked eyes. The clinical diagnosis information is obtained by various devices and technologies. For example, a nanosecond pulse near-field sensing (NPNS) technology can be used on wearable devices (such as wearable watches) to obtain blood pixel-based digital imaging information including blood cells and biochemicals flowing through blood vessels. In addition, the clinical diagnosis information is obtained by continuously flowing blood cells in 24 hours to generate the digital imaging information comprising the multiple pixel gray level changes. Moreover, the clinical diagnosis information is obtained by synchronously converting and using the clinical diagnosis information results obtained from clinical physiological and biochemical tests to obtain a biochemical and other clinical diagnosis information.

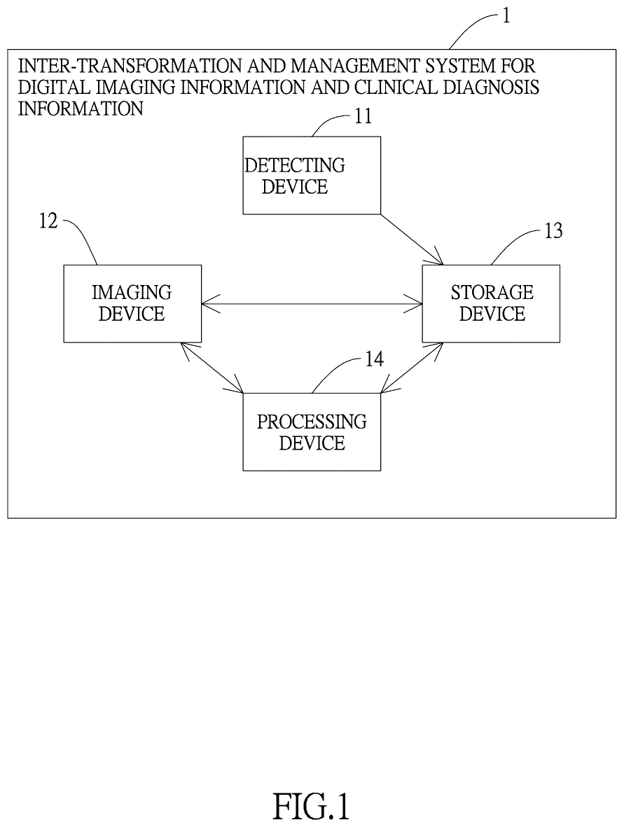

Refer to FIG. 1. FIG. 1 is the structural block schematic diagram for the inter-transformation and management system of the digital imaging information and the clinical diagnosis information. The inter-transformation and management system of the digital imaging information and the clinical diagnosis information 1 comprises a detecting device 11, an imaging device 12, a storage device 13 and a processing device (with a software program) 14. The detecting device 11 captures multiple same body part images of a human body at a predetermined sequential time period, and obtains clinical diagnosis information. In an embodiment of the invention, the detecting device 11 captures multiple same body part images of a human body at a well-defined, regular and predetermined sequential time period, and obtains clinical diagnosis information. The detecting device 11 is connected to the storage device 13. The imaging device 12 is connected to the storage device 13 to process an individual image process. The processing device 14 is both connected to the storage device 13 and the imaging device 12. The processing device 14 identifies, confirms, inter-transforms, and manages clinical diagnosis information of the imaging device 12 by a software program disposed of the processing device 14. The processing device 14 further compares and associates the clinical diagnosis information with the digital imaging information storing in the storage device 13. Therefore, the clinical diagnosis information judged by clinical medical professional's naked eye is stored as the digital imaging information by the storage device 13. The storage device 13 collects and stores the digital imaging information corresponding to the multiple sequential same body part images. The digital imaging information of the body part image comprises multiple pixels. Each pixel corresponds to each pixel gray level. The storage device 13 stores all of the digital imaging information corresponding to the individual sequential multiple same body part images. The processing device 14 persists for storing the individual clinical diagnosis information, the digital imaging information corresponding to the individual clinical diagnosis information, the pixel gray level, the changes of the pixel gray level, and the threshold point. The storage device 13 is connected to the detecting device 11, the imaging device 12, and the processing device 14. The storage device 13 stores all of the sequential individual multiple same body part images corresponding to the digital imaging information. The digital imaging information is obtained by the detecting device 11. The imaging device 12 performs the image process to facilitate the medical professionals to execute the clinical diagnose procedure by their naked eye. The processing device 14 is connected to the imaging device 12 and the storage device 13. The processing device 14 compares the digital imaging information corresponding to all of the individual sequential multiple same body part images in real time. When the clinical medical professional obtains an initial clinical diagnosis information in the multiple individual part image, the software program in the processing device 14 is executed to identify, confirm, and inter-transform the clinical diagnosis information of the disease in the same body part image by the imaging device 12 to obtain the digital imaging information corresponding to the clinical diagnosis information. Furthermore, the processing device 14 inter-transforms, manages, and compares the same position of the pixel gray level of the digital imaging information of the previous sequential multiple same body part images of the clinical diagnosis information of the individual in the well-defined, regular and predetermined time period. When the changes of the pixel gray level in the multiple digital imaging information is reaching to a threshold point, the processing device 14 generates a marked signal. The clinical diagnosis information described in the invention represents that when the same position in the sequential multiple same body part images of the individual is transferred from a normal state in an initial state to the abnormal state observed by the medical professional's naked eye, the clinical diagnosis information is obtained.

In an embodiment of the invention, the body part image comprises a fundus non-dilated pupils retinal photograph image, an X-ray image, a CT scan image, or an ultrasonic image. Each image is captured by the corresponding detecting device 11. For instance, the ultrasonic image is captured by the ultrasonic device. The CT scan image is captured by a CT scan device. Similarly, the 24-hour continuous dynamic pixel gray level changes of blood flow in the blood vessels obtained by the NPNS technology using the wearable device are able to be processed by the processing device 14 and inter-transformed and managed with the clinical diagnosis information.

In an embodiment of the invention, the detecting device 11 comprises various devices for identifying and processing images such as a fundus photography device, a radiology device, an ultrasound device, a CT scan device and other equipment. In an embodiment of the invention, both the detecting device 11 and the imaging device 12 are integrated in a same device such as an image sensor and a graphic processing unit. The detecting device 11 and the imaging device 12 are utilized to identify and process the body part image and transform the digital imaging information of the individual part stored in the storage device 13 as a body image information being widely used in the clinical diagnosis. In this way, the body image information clinically is facilitated to be examined by the medical professional's naked eye and the clinical diagnosis information is obtained by the medical professional.

In an embodiment of the invention, the storage device 13 is able to be integrated with the detecting device 11 in a same device or in a distinct device. The storage device 13 comprises a database server, a cloud server or a portable storage media device and so on. The storage device 13 is utilized to store the digital imaging information and the individual multiple pixel gray levels.

In an embodiment of the invention, the processing device 14 comprises a CPU, a digital signal processor (DSP), a micro processing unit (MPU), and a micro control unit (MCU), and so on. The processing device 14 uses the software program (Apps) to compare the pixel gray level in the same position in the digital imaging information with the same position in the sequential previous and latter clinical diagnosis information. The processing device 14 further uses the software program (Apps) to evaluate and determine whether the changing differences of the pixel gray level of the same position in each image of the previous digital imaging information and in each image of the latter digital imaging information with the same position or the same area in each image of the clinical diagnosis information is reaching to the threshold point. In the invention, the variation of the pixel gray level represents the various changing degrees of the pixel gray level of the pixel. Alternatively, the variation of the pixel gray level represents that the various number of the pixel gray level of the pixel is changing continuously. When the various changing degree or the various number of the pixel gray level of the pixel is greater than the threshold, the position of the partial human body corresponding to the pixel is determined as the disease. For instance, when the previous amount of the variation of the pixel gray level compared with the latter amount of the variation of the pixel gray level is sharply varied from 10% to 50%, the position of the partial human body corresponding to the pixel has the disease occurring and deteriorates. Alternatively, when the variation of the pixel gray level constantly raises by about 5% and reaches the threshold of 60%, it means that when the changing variation increases by 5%, the partial human body has developed the disease. Moreover, the processing device 14 comprises the processing device 14 with a cloud server, a computer in a local site, and the processing device 14 with a smartphone, but the processing device 14 is not limited thereto.

As mentioned above, the physician is able to judge and select a normal point or a normal area, a constant point or a constant area, a clear point or a clear area, and a point or an area easily to be observed and identified and never change during the whole individual's life as a reference point or a reference area. A coordinate position of a disease in the clinical diagnosis information viewed by the medical professional's naked eye according to the medical professional expertise is called a point of interest or an area of interest. The point of interest or the area of interest allows the physician to set and locate the coordinate position in the clinical diagnosis information. Furthermore, the point of interest or the area of interest is facilitated to determine the variation of the pixel gray level. In addition, the variation of the pixel gray level is generated by comparing the changing differences of the pixel gray level using the original reference point between the previous sequential multiple same body part images and latter sequential multiple same body part images at different time periods. In detail, the same point of interest (the coordinate position of the disease) or the same area of interest is obtained by using the constant reference point in each image of the previous multiple digital imaging information and the latter multiple digital imaging information by the processing device 14. Based on the constant reference point of the individual's body image, the processing device 14 determines whether the pixel gray level of the same point of interest (the coordinate position of the disease) or the same area of interest in each image of the previous multiple digital imaging information and the latter multiple digital imaging information is greater than a threshold. In other words, the processing device 14 determines whether the pixel gray level is continuously and increasingly changing in the sequential multiple digital imaging information. When the changing variation of the pixel gray levels in the previous digital imaging information and the latter digital imaging information is greater than the threshold, the processing device 14 determines that a pathology is occurring in the area of the tissue or organ of human body. When the changing differences in the gray level value of the pixel in two or more sequential digital image information obtained from the same point of interest (coordinate point position) or the surrounding area based on the original fixed reference point exceeds the threshold, the processing device 14 determines that the disease has occurred in the area of human body. At this time, multiple individual images of the human body cannot be recognized by the medical professionals to make correct clinical diagnosis with their naked eyes. Therefore, the changing variations of the pixel gray level in the sequential, multiple digital imaging information of the same individual's body part image can be obtained by the processing device 14, and the pathological changes of cells, tissues and organs in the same part of the human body can be further discovered at an early stage.

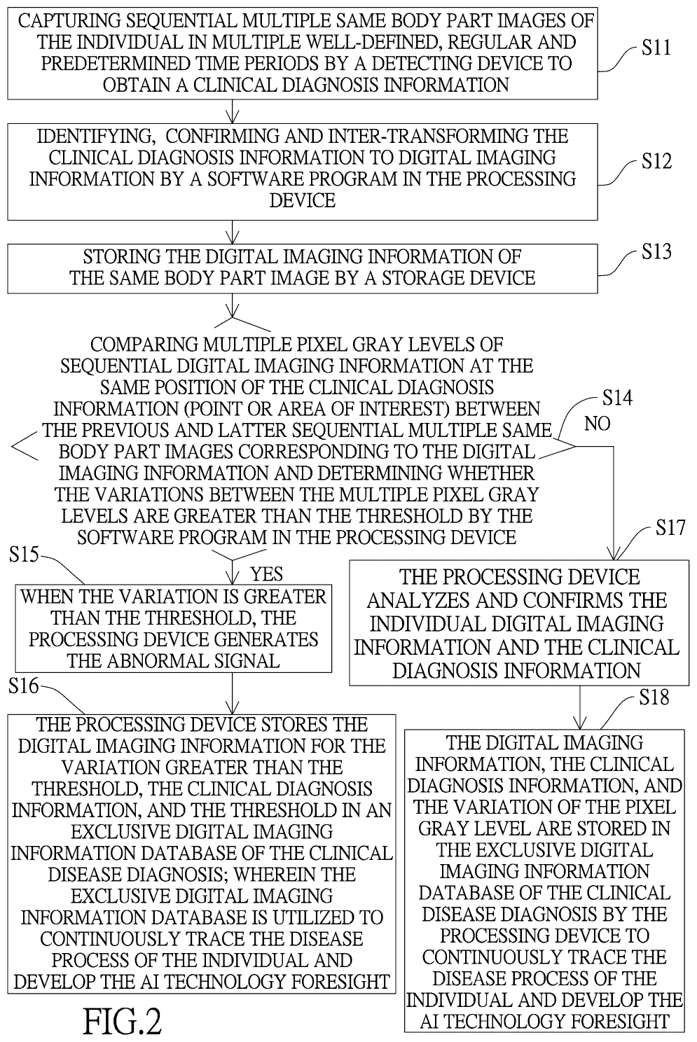

Refer to FIG. 2. FIG. 2 is the flowchart of the inter-transformation and management method of the digital imaging information and the clinical diagnosis information in the invention. The inter-transformation and management method of the digital imaging information and the clinical diagnosis information comprises the following steps: in step S11, capturing sequential multiple same body part images of the individual in sequential well-defined and regular multiple predetermined time periods by a detecting device 11 to obtain a clinical diagnosis information (the point of interest or the area of interest); in step S12, identifying, confirming and inter-transforming the clinical diagnosis information to the digital imaging information by a software program in the processing device; in step S13, storing the digital imaging information of the same body part image by a storage device; in step S14, according to the sequential time digital imaging information, comparing multiple pixel gray levels at the same position of the clinical diagnosis information (point or area of interest) between the previous and latter sequential multiple same body part images by the processing device; wherein each digital imaging information comprises multiple pixels and each pixel corresponds to a pixel gray level; furthermore, the processing device determines whether the changing variations between the sequential multiple pixel gray levels are reaching to the threshold point which is irreversible according to the individual sequential multiple digital imaging information by the software program; wherein the position of pixel gray levels of the digital imaging information corresponds to the same position of the clinical diagnosis information (point or area of interest) in the sequential previous and latter multiple same points of interest in body part image; wherein each digital imaging information comprises multiple pixels and each pixel corresponds to a pixel gray level; in step S15, when the variation is greater than the threshold, the processing device generates the marked signal; in step S16, the processing device stores the digital imaging information for the variation greater than the threshold, the clinical diagnosis information, and the threshold in an exclusive digital imaging information database of the clinical disease diagnosis. The exclusive digital imaging information database is utilized to continuously trace the disease process of the individual and develop the AI technology foresight. In step S17, when the changing variations are not reaching to the threshold point, the processing device analyzes and confirms the individual digital imaging information and the clinical diagnosis information; in S18, after the digital imaging information, the clinical diagnosis information, and the variation of the pixel gray level are analyzed and confirmed by the processing device, the digital imaging information, the clinical diagnosis information, and the variation of the pixel gray level are stored in the exclusive digital imaging information database of the clinical disease diagnosis to continuously trace the disease process of the individual and develop the AI technology foresight.

In an embodiment of the invention, the images of human body part image comprise a fundus non-dilated pupils retinal photograph image, an X-ray image, a CT scan image, or an ultrasonic image, and a nanosecond pulse near-field sensing (NPNS) technology on wearable devices. The variations of the pixel gray level generated by the continuous flow of blood in blood vessels on the surface of the human body are converted into human body physiological information, etc. In step S11, various images are captured by corresponding detecting devices. For example, the ultrasound images are captured by the ultrasound device. The CT images are captured by the CT device. The NPNS technology is used on the wearable device to capture the digital image information of blood flow in wrist blood vessels.

Each pixel of the digital imaging information corresponds to a gray level. Distinct pixel corresponds to different values of the pixel gray level. That is, distinct pixel corresponds to a different state of the pixel gray level. Therefore, in step S12 and step S14, the processing device and the software program thereof compare multiple pixels of the same body part image corresponding to the pixel gray level; wherein multiple pixels of the same body part image are stored in the storage device. For the image represented by the pixel gray level of 8 bits, the range of the pixel gray level is between 0 and 255 and the image is represented by 8 bits. Furthermore, as the formula: 2\hat{ }8=256, the imaging device is able to illustrate 256 types of the gray level, that is, 00000000 (black color) to 11111111 (white color). In another embodiment, when the body part image needs to be illustrated by higher resolution, the imaging device with higher bits can be used to represent more colors with the more pixel gray levels. The imaging device with higher bits comprises an imaging device with 16 bits, 32 bits, or higher bits to represent more pixel gray levels. In the embodiment of the invention, the bits of the imaging device for representing the pixel gray level are not limited to 8 bits.

In step S14, the processing device utilizes the software program to compare the pixel gray levels corresponding to the constant reference point and area with the same coordinate position in the previous and latter sequential multiple digital imaging information stored in the storage device. The processing device compares the pixel gray levels at the point of interest with the same coordinate position in previous and latter individual sequential multiple digital imaging information according to the clinical diagnosis information (point or area of interest) and the corresponding digital imaging information. After comparing, the processing device determines whether the changing variation between the pixel gray levels is greater than the threshold. By determining the variation between the pixel gray levels, the processing device precisely determines that the point or area of interest in the body part image corresponding to the pixel gray level develops abnormal pathological changes. Accordingly, the processing device generates the marked signal. In other words, in step S14, the processing device 14 inter-transforms the clinical diagnosis information to the digital imaging information. The processing device 14 further compares multiple pixel gray levels in the digital imaging information corresponding to the same position of the clinical diagnosis information in the previous and latter sequential multiple individual part images. After comparing, the processing device is able to determine whether the variation between the pixel gray levels of sequential multiple digital imaging information is greater or reaching to the threshold and performs the next steps S15 and S16 or performs the next steps S17 and S18.

In addition, the processing device determines whether the variation between the multiple pixel gray levels at the same position of the individual digital imaging information at different time periods is greater than the threshold. The pixel is a minimum unit in the image. Hence, in another embodiment, the processing device determines whether the variation between the blocks comprising multiple pixel gray levels at the same position of the individual digital imaging information at different time periods is reaching to the threshold point. When the variation between the multiple pixel gray levels or the variation between the blocks comprising multiple pixel gray levels is reaching to the threshold point, the processing device generates the marked signal. Moreover, in general, when the clinical diagnosis information of the individual is obtained at a first time, then the digital imaging information of the disease would be worked out by inter-transforming the clinical diagnosis information of the individual at the first time. The processing device, based on the digital imaging information obtained at the first time, determines whether the changing variation between the pixel gray levels is greater than the threshold. Alternatively, the processing device uses the first digital image information as the comparison basis in subsequent comparisons. In fact, according to the clinical judgment of the professional physicians, the digital image information of the individual's consecutive images at different time periods can be accordingly inter-transformed and connected with the digital imaging information of the individual's possible clinical diagnosis information at the same position at different time periods as the comparison basis.

Furthermore, in an embodiment of the invention, the processing device integrates an AI technology, an image recognition software, and a database technology to analyze and determine whether the variation between the pixel gray levels is greater than the threshold, i.e., machine learning technology. Moreover, in practice, when the processing device generates the result according to aforementioned steps, the digital imaging information, the clinical diagnosis information, and the threshold are stored in the processing device to build the exclusive digital imaging information database corresponding to various clinical disease diagnoses. Via the AI technology, the processing device is capable of actively learning, analyzing, and determining the result according to the digital imaging information, the clinical diagnosis information, and the threshold judged by the physician and stored in the exclusive digital imaging information database.

In below figures, the application takes the diabetes fundus retinal vasculature pathological changes as an example. In other embodiments, the examples comprise a mammography examination, a chest and abdominal radiography examination, a neck thyroid examination, an abdomen and pelvic ultrasound examination, a brain CT scan examination, and other various examinations that can obtain images. Furthermore, the examples comprising the nanosecond pulse near-field sensing (NPNS) technology can be used on wearable devices (such as wearable watches) to continuously obtain the dynamic changes of the pixel gray level of the blood stream flowing through blood vessels in 24 hours. The examples are described as below embodiments.

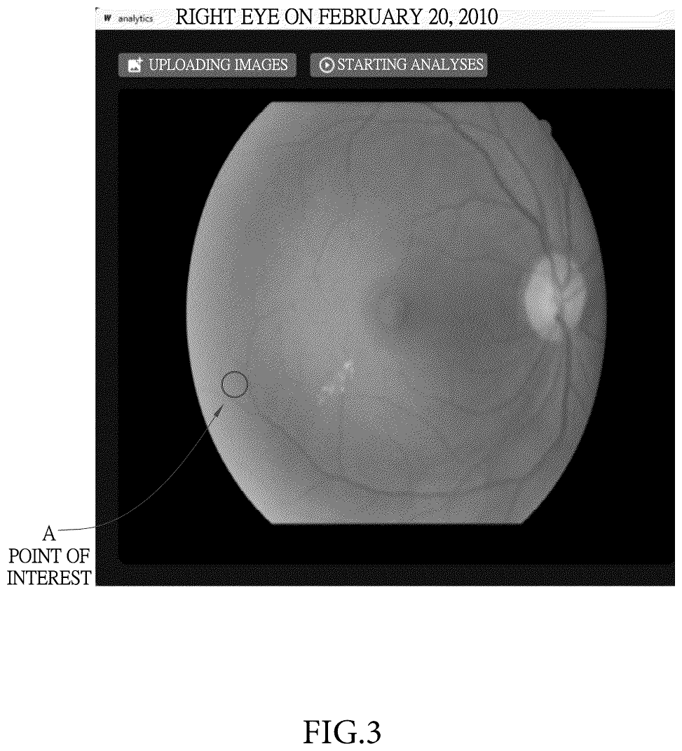

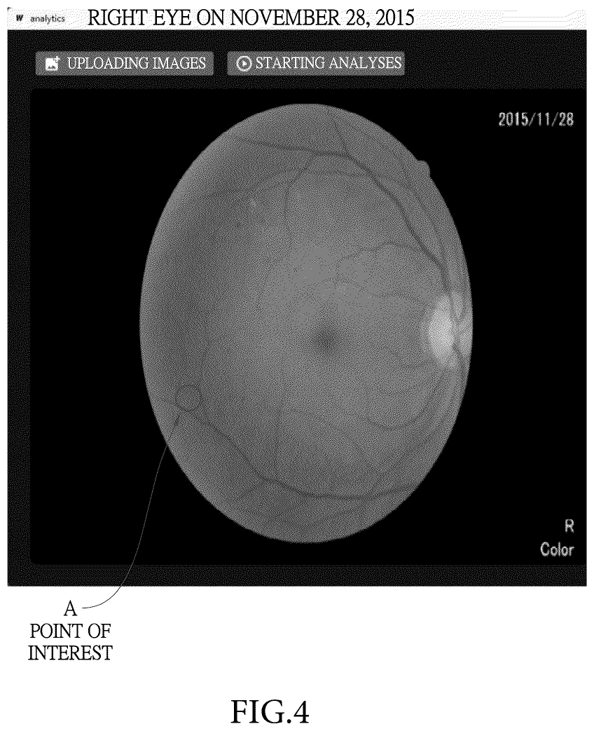

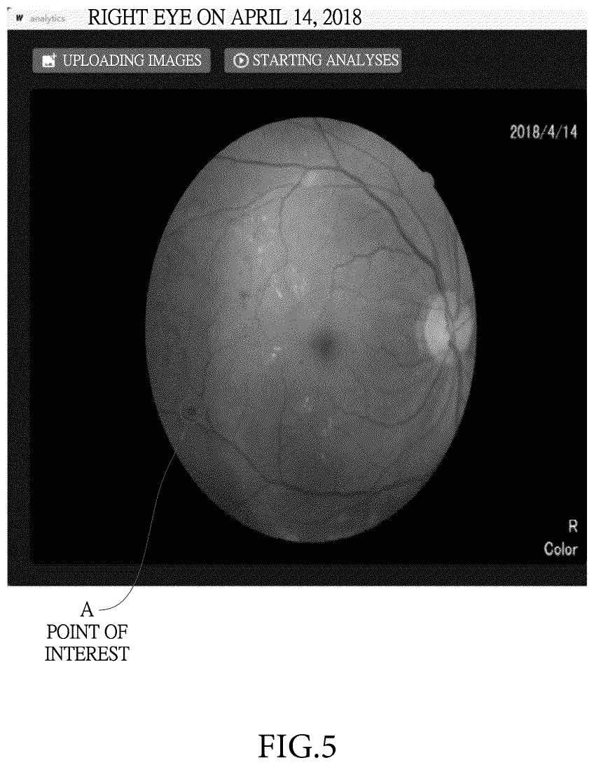

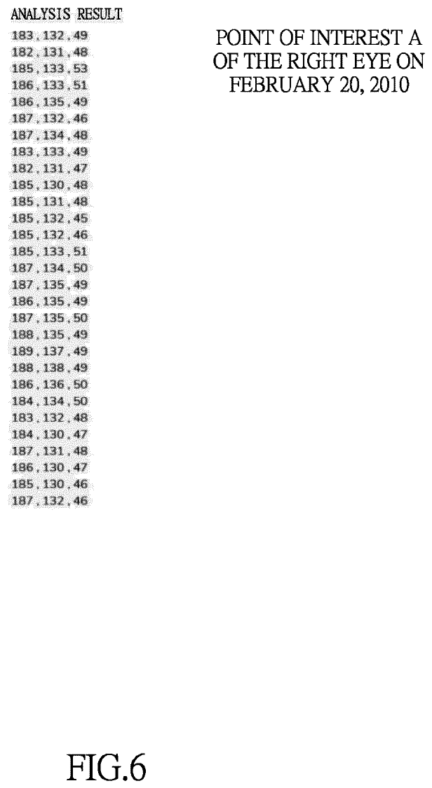

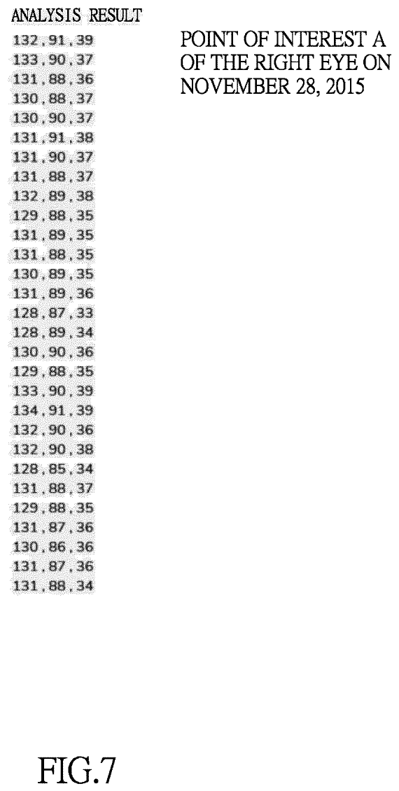

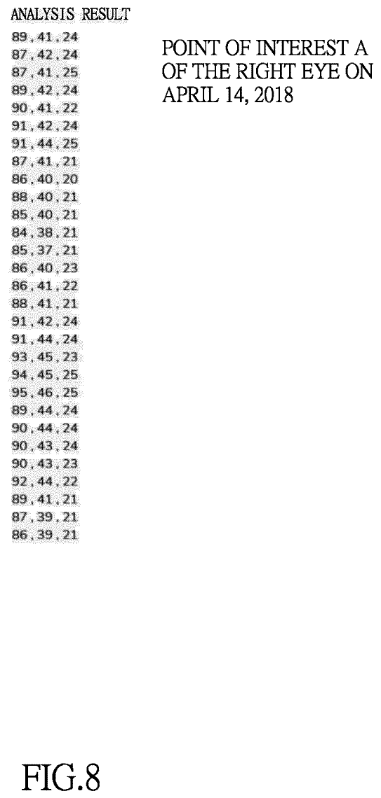

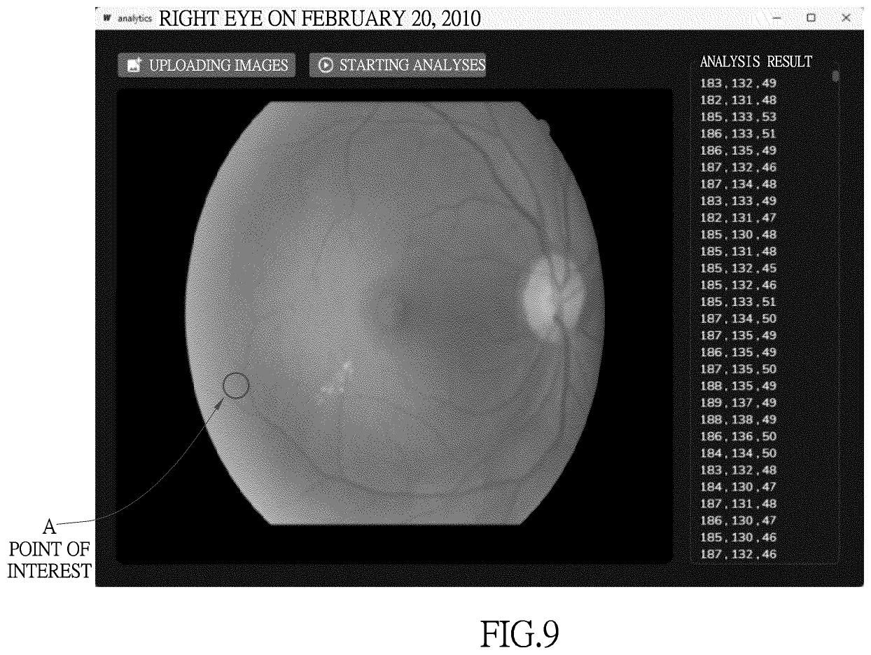

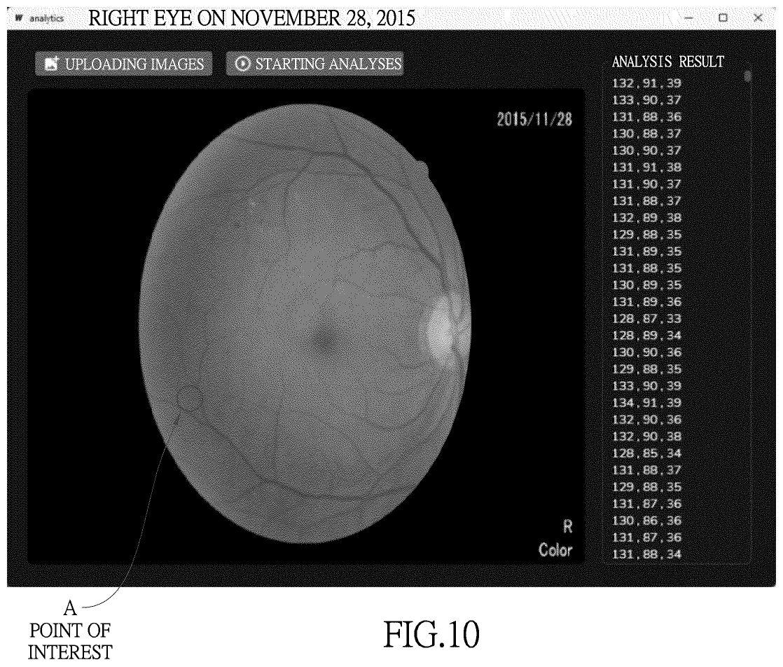

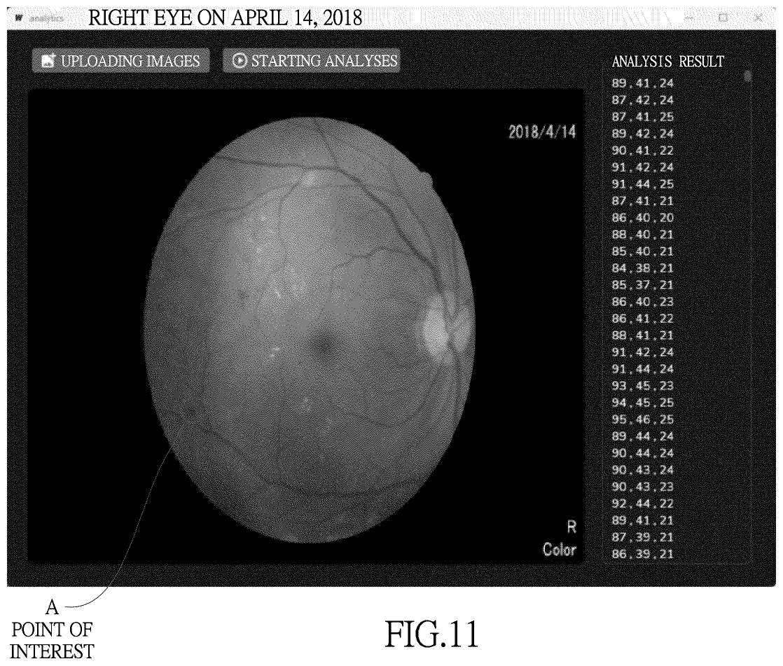

Refer to FIG. 3 to FIG. 5. FIG. 3 to FIG. 5 are the sequential clinical diagnosis information diagrams at distinct periods for the right eye of the diabetes non-dilated pupil fundus retinal vasculature in the invention. FIG. 3 was shot on Feb. 20, 2010. FIG. 4 was shot on Nov. 28, 2015. FIG. 5 was shot on Apr. 14, 2018. The symbol A in the figure represents the range of the point of interest or the area of interest. The point of interest A or the area of interest A in FIG. 5 is the position of the initial clinical diagnosis information obtained by the medical professional with the naked eye. FIG. 6 to FIG. 8 are the pixel gray levels of the point of interest A or the area of interest A corresponding to the position of the fundus retinal. FIG. 6 is the analysis result of the pixel gray level on Feb. 20, 2010. FIG. 7 is the analysis result of the pixel gray level on Nov. 28, 2015. FIG. 8 is the analysis result of the pixel gray level on Apr. 14, 2018. FIG. 9 to FIG. 11 are the schematic diagrams of the pixel gray level for the processing device utilizing the software program to illustrate the position of the fundus retinal vasculature pathological changes and the around area of the point of interest A. FIG. 9 is the analysis result of the pixel gray levels and the clinical diagnosis information on Feb. 20, 2010. FIG. 10 is the analysis result of the pixel gray levels and the clinical diagnosis information on Nov. 28, 2015. FIG. 11 is the analysis result of the pixel gray levels and the clinical diagnosis information on Apr. 14, 2018. FIG. 5 is the position of the retinal vasculature pathological changes observed by the clinical professional's naked eye on Apr. 14, 2018 at the first time. FIG. 5 is the clinical diagnosis information. The point of interest A or the area of interest A is disposed in the clinical diagnosis information. The software program in the processing device identifies, confirms, inter-transforms and manages the point of interest A or the area of interest A in the clinical diagnosis information to correspond to the digital imaging information. The point of interest A or the area of interest A of the digital imaging information comprises multiple pixel gray levels. The multiple pixel gray levels are the digital imaging information. After transformed, a part of the digital imaging information is illustrated as FIG. 6 to FIG. 8, which can be detected to be illustrated.

As shown in FIG. 3 to FIG. 5, when the patient is diagnosed by the non-mydriatic fundus retina photograph on Apr. 14, 2018 to generate the clinical diagnosis information, the physician can hardly observe whether the position around the point of interest A in the fundus part image generates the pathological changes by comparing the previous fundus part image in FIG. 3 and the latter fundus part image in FIG. 4 with the naked eye.

Refer to FIG. 6 to FIG. 8 again. FIG. 6 is the analysis result of the pixel gray level on Feb. 20, 2010. FIG. 7 is the analysis result of the pixel gray level on Nov. 28, 2015. FIG. 8 is the analysis result of the pixel gray level on Apr. 14, 2018. According to the analysis result of the pixel gray level in FIG. 6, the values of the pixel gray level are approximate to the values of 185, 130, and 45. Five years later, according to the analysis result of the pixel gray level generated on Nov. 28, 2015 in FIG. 7, the pixel gray levels have been significantly varied. The values of the pixel gray level are approximately changed from the values of 185, 130, and 45 to the values of 130, 88, 35. The variations exceed the threshold of the pixel gray level. However, in the meanwhile, the physician fails to compare the clinical diagnosis information in FIG. 3 with FIG. 4 by the naked eye to clearly identify around the point of interest of the fundus retinal may have the pathological changes occurring. FIG. 8 shows the analysis result of the pixel gray level generated at the period of Apr. 14, 2018, the pixel gray levels have been varied from the values of 130, 88, and 35 to the values of 90, 45, and 22. In the meanwhile, the physician is able to compare the clinical diagnosis information in FIG. 5 with the digital imaging information in FIG. 8 by the naked eye. After comparing, the physician discovers that the fundus retinal vasculature around the point of interest or the area of the interest incurred the pathological changes of the initial clinical diagnosis information. Therefore, diseases are able to be detected by determining the variation of the pixel gray level in the digital imaging information in the invention to diagnose and cure in an early stage.

Refer to FIG. 9 to FIG. 11. FIG. 9 to FIG. 11 are the schematic diagrams of the processing device utilizing the software program to illustrate the pixel gray level around the point of interest A of the fundus retinal vasculature pathological changes. In FIG. 9, FIG. 10, and FIG. 11, the processing device 14 utilizes the software program to inter-transform and manage the clinical diagnosis information to correspond to the digital imaging information. Furthermore, the processing device 14 analyzes the pixel gray level in the clinical diagnosis information. The processing device 14 in the invention uses the software program to rapidly, precisely, and instantly identify, confirm, and inter-transform the clinical diagnosis information as the digital imaging information corresponding to the clinical diagnosis information. After inter-transformed and managed, the pixel gray level in the digital imaging information corresponding to the sequential multiple individual image of the clinical diagnosis information can be applied the diagnosis and the treatment in the early stage.

As mentioned above, the inter-transformation and management system of the digital imaging information and the clinical diagnosis information and the method thereof determine that the individual has the pathological changes occurring at the point of interest or the area of interest in the clinical diagnosis information. Accordingly, the time period of the pathological changes can be derived from the early stage. In addition, the digital imaging information and the clinical diagnosis information of the multiple individuals are collected and stored as the aforementioned database to build various disease prediction models. In other words, the inter-transformation and management system of the digital imaging information and the clinical diagnosis information and the method thereof utilize the above steps to collect and store the individual digital imaging information, to form the group digital imaging information, and to build the digital imaging database. The system and the method of the invention use the group digital imaging database to train the AI. The various disease prediction models can be built by the AI and widely applied to group healthcare.

In the meanwhile, the processing device utilizes the software program to analyze and compare the fundus retinal vasculature pathological changes in the clinical diagnosis information and the fundus retinal vasculature pathological changes in the digital imaging information of the diabetes patient at the same position stored in the storage device. The processing device utilizes the software program to build the inter-transformation according to the clinical diagnosis information and the digital imaging information of the fundus retinal vasculature pathological changes for the same subject in the real world. The software program of the processing device identifies, confirms, inter-transforms, and manages the clinical diagnosis information and the digital imaging information of the fundus retinal vasculature pathological changes to confirm the corresponding relative between the clinical diagnosis information and the digital imaging information for the individual fundus retinal vasculature pathological changes. After that, the software program compares and determines the same coordinates and the same areas in all the same non-mydriatic fundus retina photographs of the individual in the sequential previous and later periods to obtain the variation of the pixel gray level at the point of interest or the area of interest A of the clinical diagnosis information in distinct periods.

Moreover, by looking back at the variations in the digital imaging information pixel gray level of the same interest point or area in the clinical diagnosis information of the non-mydriatic fundus retinal photography taken each year in a continuous and chronological order before the patient was diagnosed with the fundus retinal microvascular disease by the professional physician's naked eye, it can be found and confirmed that even before the clinical diagnosis information of vascular abnormalities of the fundus retinal microvascular disease can be seen by the naked eye, the pixel gray levels at the same position corresponding to the point of interest or the area of interest in the initial clinical diagnosis information have already shown abnormal digital imaging information changes. This is far earlier than the period when the clinical professional physician first diagnosed the patient with the diabetic fundus retinal microvascular disease with the naked eye.

In contrast, according to the non-mydriatic fundus retina photograph of the diabetes patient in sequential well-defined and regular time period each year, it shows that the pixel gray levels of the digital imaging information in any normal point or area of interest corresponding to the simulating clinical diagnosis information are the same from year to year without significant changes. The same result is shown even among different individuals, races, or ethnic groups. Therefore, the annual non-mydriatic retinal photography examination examined in chronological order and looking back at the digital imaging information when the pixel gray level of any position of the retina changes abnormally (perhaps this can be regarded as a sign of the development of the early clinical diagnosis information based on the computer digital imaging information) and the clinical diagnosis information seen by the clinician physician with the naked eye, the identification, the confirmation, and the inter-transformation between the digital imaging information and the clinical diagnosis information will become the method of non-mydriatic retinal photography examination for the initial and earliest accurate clinical diagnosis of diabetic retinal microvascular disease, and will also become the basis for the application of artificial intelligence in the early diagnosis and treatment of diabetic retinal microvascular disease.

It should be noted that in above embodiments, the values of the pixel gray level and the threshold are examples but are not limiting. That is, in the real world of the clinical practice, the variation of the pixel gray level and the threshold in the digital imaging information generated by inter-transforming the clinical diagnosis information are determined and defined according to distinct physicians.

Each clinical individual's ray image information comprises a thoracic cavity and abdomen X-ray photograph, a magnetic resonance imaging, a CT scan, and a neck ultrasonic scanning. Furthermore, the digital imaging information comprises a thyroid ultrasonic scanning, a breast ultrasonic scanning, a pathology stain slide glass of cell tissue information, bio-materials flowing in a wrist vessel obtained by utilizing the NPNS technology applied to a wrist wearable device, and a structure composition of a blood cell (A clinical biochemistry/blood cell information is obtained by inter-transforming and managing the variation of the multiple pixel gray levels and the clinical detection diagnosis). Then each clinical individual's ray image information is inter-transformed and managed by the detecting device, the imaging device, the processing device with the software program. On the other hand, the detecting device, the imaging device, the processing device, and the software program thereof persistently inter-transform and manage the digital imaging information and the clinical diagnosis information stored in the storage device. Furthermore, the detecting device, the imaging device, the processing device, and the software program thereof persistently inter-transform and manage each clinical individual's ray image information, the digital imaging information and the clinical diagnosis information stored in the storage device. The procedure and the results such as the threshold point are stored by the processing device. In contrary to the conventional technology of the detecting device for utilizing a radiant ray and an ultrasound to generate a computer color photograph corresponding to the clinical diagnosis information, the method of the inter-transformation and the management between the individual digital imaging information arranged in sequence and the clinical diagnosis information observed by the physician's naked eye in the invention will become a new clinical diagnosis in medicine. The method provides the effective and precise diagnosis and treatment in the early stage and facilitates to develop the AI technology in the early stage.

In addition, the aforementioned individual digital imaging information and clinical diagnosis information have the same presentation even in different human ethnicity. That is, no matter what kinds of the computer detecting devices/equipment be utilized for detecting the same human organ and the same tissue, the visible imaging appearance shows almost the same including both the clinical diagnosis information and the digital imaging information (pixel gray level). Actually, when an organ and a tissue is represented as normal in the original normal image by professional's naked eye review, but abnormal in the digital imaging information, the physician would not be able to judge that the organ and the tissue in the original normal image have evidence of abnormal. That is, the human organ and the tissue in viewing of the digital imaging information are regarded would be an earliest and great significant and especially in a high risk disease entity in the clinical diagnosis settings. Obviously, when the same organ and the same tissue of the human body is objectively detected by the same detecting device, the digital imaging information (pixel gray level) is the same. In summary, whatever normal tissue, normal organ, abnormal tissue, or abnormal organ are examined by using any detecting device/equipment, the digital imaging information and clinical diagnosis information would be the same for the same organ and the same tissue even in different ethnicity.

In summary, for the research in the development and the application of the artificial intelligence technology in the clinical disease diagnosis and treatment, the detection devices such as radiation, ultrasound or computer with photograph, the imaging devices, the digital image information stored in the storage devices and the processing devices, and the radiotherapy equipment are connected through the Internet, so that the clinical diagnosis (based on the difference in pixel gray level changes in the digital image information) and treatment methods (radiotherapy/high-energy electron/magnetic wave ablation therapy) are combined into one. Based on today's wireless network technology, the inter-transformation and management of the pixel digital image information would be widely used. Through the artificial intelligence and the machine learning, accurate clinical diagnosis would be very early performed. According to the earliest clinical diagnosis, the individual is immediately and automatically given the correct, accurate and effective treatment in real time, thereby promoting the early diagnosis and early treatment of diseases so that precision medicine can be achieved.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. An inter-transformation and management method for digital imaging information and clinical diagnosis information, comprising the following steps:
capturing multiple same body part images of a human body at each predetermined sequential time period, and obtaining clinical diagnosis information according to the same body part images by a detecting device;
processing an individual image process for obtaining the clinical diagnosis information by an imaging device;
storing individual digital imaging information by a storage device;
automatically identifying, confirming, and inter-transforming the clinical diagnosis information to the digital imaging information by a software program of a processing device; wherein the digital imaging information comprises multiple pixels, and each of the pixels corresponds to a pixel gray level; and
automatically determining whether any changing differences between the pixel gray levels of the pixels at a same position of the sequential multiple digital imaging information of the clinical diagnosis information in any one of the individual digital imaging information is reaching to a threshold point by the software program of the processing device;
wherein when any one of the changing differences is greater than the threshold point, the processing device generates a marked signal.

2. The inter-transformation and management method for the digital imaging information and the clinical diagnosis information as claimed in claim 1, further comprising a step of:
storing the same body part images of the human body of the digital imaging information of the clinical diagnosis information by the storage device.

3. The inter-transformation and management method for the digital imaging information and the clinical diagnosis information as claimed in claim 1, wherein each of the same body part images of the human body comprises a fundus non-dilated pupils retinal photograph image, an X-ray image, a computed tomography (CT) scan image, and a nanosecond pulse near-field sensing image in a wearable device, or an ultrasound image.

4. The inter-transformation and management method for the digital imaging information and the clinical diagnosis information as claimed in claim 1, wherein the processing device determines whether the changing differences between the pixel gray levels of the pixels at a same reference point of the digital imaging information of the clinical diagnosis information and the individual digital imaging information is reaching to the threshold point by the software program of the processing device;
wherein the reference point corresponds to the same position of the pixels of the digital imaging information of the clinical diagnosis information and the individual digital imaging information.

5. The inter-transformation and management method for the digital imaging information and the clinical diagnosis information as claimed in claim 1, wherein the processing device builds an artificial intelligence (AI) image recognition software to automatically compare the individual digital imaging information in real time with the digital imaging information of the clinical diagnosis information for determining the differences or generating the marked signal.

6. The inter-transformation and management method for the digital imaging information and the clinical diagnosis information as claimed in claim 1, wherein the detecting device captures the multiple same body part images of the human body at each well-defined, regular, and predetermined sequential time period, and obtaining the clinical diagnosis information according to the same body part images.

7. An inter-transformation and management system for digital imaging information and clinical diagnosis information, comprising:
a detecting device, capturing multiple same body part images of a human body at each predetermined sequential time period, and obtaining clinical diagnosis information according to the same body part images;
an imaging device, connected to the storage device to process an individual image process for obtaining the clinical diagnosis information;
a storage device, connected to the detecting device and the imaging device, and storing individual digital imaging information, the multiple same body part images, and multiple pixel gray levels at each predetermined sequential time period; and
a processing device with a software program thereof, connected to the imaging device and the storage device, obtaining the clinical diagnosis information in real time, identifying, confirming, and inter-transforming the clinical diagnosis information to the digital imaging information by the software program, and determining whether any changing differences between pixel gray levels of pixels at a same position of the digital imaging information in any one of the individual digital imaging information is reaching to a threshold;
wherein when the changing differences in any one of digital imaging information is reaching to the threshold, the processing device generates a marked signal.

8. The inter-transformation and management system for the digital imaging information and the clinical diagnosis information as claimed in claim 7, wherein the storage device stores the same body part images of the human body of the digital imaging information of the clinical diagnosis information by the storage device.

9. The inter-transformation and management system for the digital imaging information and the clinical diagnosis information as claimed in claim 7, wherein each of the same body part images of the human body comprises a fundus non-dilated pupils retinal photograph image, an X-ray image, a computed tomography (CT) scan image, and a nanosecond pulse near-field sensing image in a wearable device or an ultrasonic image.

10. The inter-transformation and management system for the digital imaging information and the clinical diagnosis information as claimed in claim 7, wherein the processing device determines whether the differences between the pixel gray levels of the pixels at a same reference point of the digital imaging information of the clinical diagnosis information and the individual digital imaging information is reaching to the threshold by the software program of the processing device;
wherein the reference point corresponds to the same position of the pixels of the digital imaging information of the clinical diagnosis information and the individual digital imaging information.

11. The inter-transformation and management system for the digital imaging information and the clinical diagnosis information as claimed in claim 7, wherein the processing device builds an artificial intelligence (AI) image recognition software to automatically compare the individual digital imaging information in real time with the digital imaging information of the clinical diagnosis information for determining the changing differences or generating the marked signal.

12. The inter-transformation and management system for the digital imaging information and the clinical diagnosis information as claimed in claim 7, wherein the detecting device captures the multiple same body part images of the human body at each well-defined, regular, and predetermined sequential time period, and obtaining the clinical diagnosis information according to the same body part images.
